(19) United States
(12) Patent Application Publication
(10) Pub. No.: US 2003/0093296 A1
(43) Pub. Date: May 15, 2003

(54) Method and system of managing information for a hospital

(76) Inventors: Dong Hyounk Lee, Seoul (KR); Young Sunk Kim, Seoul (KR); Jin Young Lee, Seoul (KR); Jin Young Ann, Seoul (KR); Dong Ook Kim, Seoul (KR)

Correspondence Address: Kang Nam, G W i P S, P.O. Box 2301, Seoul, 135-621, KR

(21) Appl. No.: 10/165,071

(22) Filed: Jun. 10, 2002

(30) Foreign Application Priority Data
Nov. 10, 2001 PCT/KR01/01705

(51) Int. Cl. G06F 17/60; A61B 1/00; G06F 7/00; G06F 17/00; G06K 9/00; G06K 9/46; A61B 5/00

(52) U.S. Cl. 705/2; 705/3; 707/104.1; 600/300; 382/232; 382/128; 600/101

(57) ABSTRACT

A system and method for managing information of a hospital, which can facilitate information exchange between an order communication system and an information management system by designing information fields respectively established on the two systems. The system for managing information of the hospital includes an interface module for providing inter-operability with respect to the information exchange between the information management system and the order communication system; a data interchanging unit for inducing patient information, examination information and reservation information to be reviewed through the respective systems via the interface module; and a result-inputting unit for storing the review result processed by the data interchanging unit in databases of the information management system and the order communication system. The method for managing information for a hospital includes the steps of transmitting information from the order communication system to the information management system through the interface module; and integrating a process of transmitting information from the information management system to the order communication system. The step of transmitting information from the order communication system to the information management system includes designing a patient information message, an examination information message, and a medical treatment reservation message and examination reservation information system to transmit patient reservation results. Accordingly, the invention has the advantage of ensuring reliability and accuracy in information and stability within the system.

### BACKGROUND OF THE INVENTION

1. Field of the Invention

The present invention relates to a method and system for managing information of hospital. Especially, the order communication system and information management system are designed to cross-link and cross-utilize their respective databases in order to derive relational commonalities of information between their respective data fields, will result in an easy exchange of information between the systems.

2. Description of the Prior Art

Information collected and processed in a hospital requires systematic and integrated management. The quantity of information rapidly expands in proportion to the number of patients. This information includes information obtained from the patients as well as information already stored on the hospital's database. A critical factor in the care and ultimate healing of patients is the hospital's ability to acquire and deliver timely and accurate images and personal record information and images to the hospital staff.

FIG. 1 illustrates the workflow in a hospital, through which collection and application of information can be tracked. The work flow includes a photographing and diagnosis step S10 consisting of image acquisition 10, inspection 11, interpretation 12 and transcription 13, a medical examination step S20 of providing information acquired from the photographing and diagnosis step S10 to a ward 20, an outpatient clinic 21, an operating room 22, an emergency room 23 and a prescription part 24 or feeding back the information, and an information application step S30 of using the information obtained from the medical examination step S20 for a meeting/routine round 30, research file 31 and education 32.

Images of a patient are obtained using an image acquisition device such as a microscope, an endoscope, a medical photograph and a motion-picture apparatus. These images are stored in files or as scanned images within a database using compression storage methods such as layered compression using difference pulse code modulation. These files or scanned images can be viewed on-line or stored off-line for later use and would provide supporting information in the medical examination of the patient.

In the medical examination step or information application step, the image information obtained from the patient is compression-stored in the database and employed by an authorized manager (doctor) for healing the patient in real-time. Accordingly, accuracy and reliability of various information items on the database constructed in the hospital are considerably important. Therefore, the medical examination step can employ the information in real-time in cases where integrated processing of information is executed and inter-operability of various data items is maintained.

FIG. 2 illustrates system resources constructing an information management system 40, which are connected on a network, showing information exchange between the information management system 40 and an order communication system 50. The information management system 40 includes: an image information acquisition means 80 consisting of an image photographing device 60 corresponding to an imaging apparatus and an image control and compression device 70 for controlling the image photographing device 60, realizing data obtained by the image photographing device as images and compressing image data if required; a host computer system 100 for inputting image information collected through the image information acquisition means 80 into a database 90 and transmitting corresponding information at the request of a user; an image display 110 for requesting the host computer system 100 for image data and combining or laying out received images; a monitor 120 for monitoring an image made by the image display 110; and a web server 140 for transmitting data processed by the host computer system 100 to a user terminal 130 through the Internet. The information management system 40 has been designed for delivering optimized information that satisfies user environments.

In general, information employed by the information management system 40 includes image information, examination information, patient information and interpretation information. Image information fields construct an examination acquisition date, an examination acquisition time, examination execution date, a photographing room number, an examination execution time, an examination item code, an examination item name, a photographing system, a patient ID, a patient name, etc. Examination information fields include a patient's ID, name, date of birth and sex as well as the name of the ordering doctor and the main physician. Also included is an order issuing ward, an order issuing department, an examination item code, an item related to cancellation of examination, an examination room code, requirements/complaints, an examination application date/time, classification into ward and outpatient and an order serial number, etc.

Patient information fields construct a patient's ID, name, date of birth, sex, ward, medical department, chief physician, classification, social security number and any other relevant information. Interpretation information fields construct a temporary interpretation date, a temporary interpretation time, a transcription date/time, a formal interpretation date/time, a temporary interpretation doctor, a transcriber's ID, a transcription classification code, transcription processing conditions, a patient's ID, the location of a voice interpretation file, a conclusion, a text and other relevant information. The information fields transmitted from the information management system 40 providing the image information, examination information, patient information and interpretation information are not processed according to data processing but simply serve as an image viewer. That is, when a user requests the host computer system 100 of the information management system 40 for information data through the user's terminal 130 or image display 110 and monitor 120 via a short-distance/long-distance communication network or the Internet, only the retrieval function that the data is transmitted through on-line not off-line is provided.

The order communication system 50 updates patient information. Hospital staff retrieves the updated information stored in the order communication system 50. This method does not support patient information, examination information, reservation information and interpretation information required by the hospital, especially, diagnosis radiology, or a user such as a clinician. Furthermore, it cannot display information in a timely manner, but rather, simply reviews and retrieves only information in the order communication system. Additionally, since it does not carry information via the database of the information management system, its stability and efficiency are deteriorated. Finally, all user systems are concentrated on the order communication system 50 resulting in a decrease in information reliability due to network overload or system downtime.

Consequently, the conventional information management system and the order communication system cannot exchange required information in a timely manner between each other. Moreover, all systems in the hospital can access only the database of the order communication system. This increases the possibility of an overload to the network, burdening its performance, resulting in the erroneous operation of the order communication system, degrading its reliability and stability.

### SUMMARY OF THE INVENTION

Accordingly, an object of the present invention is to provide an interface module between the information management system and order communication system, by which the information management system and order communication system constructed in a hospital can exchange information with each other at a precise point of time.

Another object of the present invention is to eliminate causes of network overload due to the saturation of requests and information queries concentrated on the order communications system's database.

Still another object of the present invention is to prevent data error in the order communication system and improve its operation reliability.

To accomplish the objects of the present invention, an integrated solution is provided, delivering a unified hospital information management system comprised of an order communication system and an information management system. These systems will be connected on a network, the information management system includes: a means of acquiring image information with an image photographing device corresponding to an imaging system and an image control and compression device for controlling the image photographing device, realizing data obtained from the image photographing device and compressing image data if required; and a host computer system for inputting image information into a database through the image information acquisition means, the order communication system including an image display that requests the host computer system for image data and combines or lays out images sent from the host computer system, a monitor for monitoring the image made by the image display, and a web server for transmitting data processed by the host computer system to a user terminal via the Internet, the integrated hospital information management system comprised of (1) an interface module providing inter-operability with respect to information exchange between the information management system and the order communication system, (2) a data interchanging unit for inducing examination information and patient information, which are generated from examination images and stored in the database of the information management system, interpretation information attained from the examination images, and patient information, examination information and reservation information to be reviewed through the respective systems via the interface module, and (3) a result inputting unit for storing the reviewed result processed by the data interchanging unit in the databases of the information management system and the order communication system.

To accomplish the objects of the invention, it is also provided an integrated hospital information management method for maintaining compatibility and permitting exchanging and sharing of various information items including patient information, image information, reservation information and examination information, which exist on each of the order communication and an information management systems, the three-step method including (1) transmitting information from the order communication system to the information management system and integrating a process of transmitting information from the information management system to the order communication system, (2) transmitting information from the order communication system to the information management system comprised of: a patient information message designing step of changing an access path of patient images according to admission/discharging of patients and which varies a patient searching path according to department transfer/ward transfer; an examination information message designing step of, according to the path of information required for matching the photographed image of a patient and corresponding examination between the order communication system and the information management system where quality control check of the information is performed and a feedback message is given indicating the probability of a successful match, a medical treatment reservation message designing step and an examination reservation information designing step of, when a reservation of patient has been completed, setting it to a point of time at which the patient information message and examination information message are transmitted and, when the reservation is cancelled, delivering the result, (3) the process of transmitting information from the information management system to the order communication system being carried out in such a manner that interpretation information is transmitted, with reports on examinations and corresponding images matching with the corresponding examinations and images, having creation, up-date and confirmation as points of time of information transmission.

### BRIEF DESCRIPTION OF THE DRAWINGS

Further objects and advantages of the invention can be more fully understood from the following detailed description taken in conjunction with the accompanying drawing in which:

FIG. 1 illustrates a workflow in information management and application steps in a hospital.

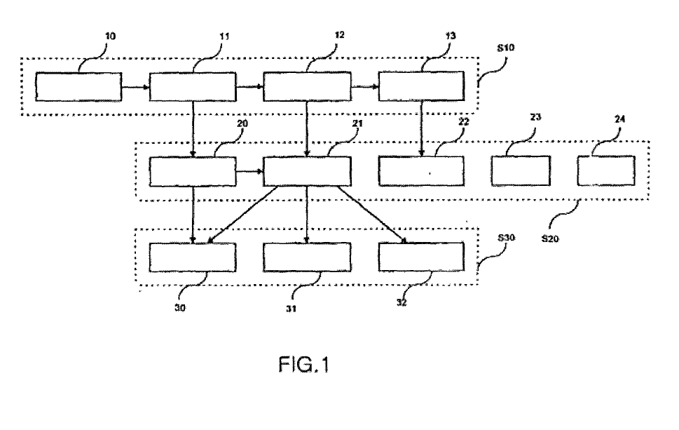

FIG. 2 illustrates an example of the configuration of an on-line information management system in a hospital.

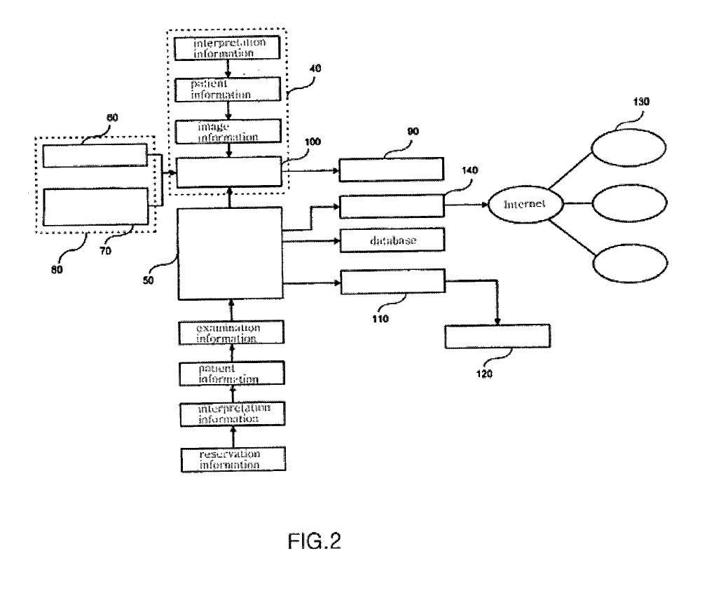

FIG. 3 illustrates the configuration of a system according to the present invention.

FIG. 4 illustrates an essential part of the system according to the present invention.

FIG. 5 illustrates an embodiment of the system according to the present invention.

FIG. 6 illustrates a message information processing flow according to the interface module of the present invention.

FIG. 7 illustrates an information processing sequence according to the present invention.

FIG. 8 illustrates a modeling of hospital works for analysis of the hospital works.

FIG. 9 is a sequence diagram showing order, admission and discharging and transfers processes with the lapse of time, processed by the present invention.

### DETAILED DESCRIPTION OF A PREFFERRED EMBODIMENT

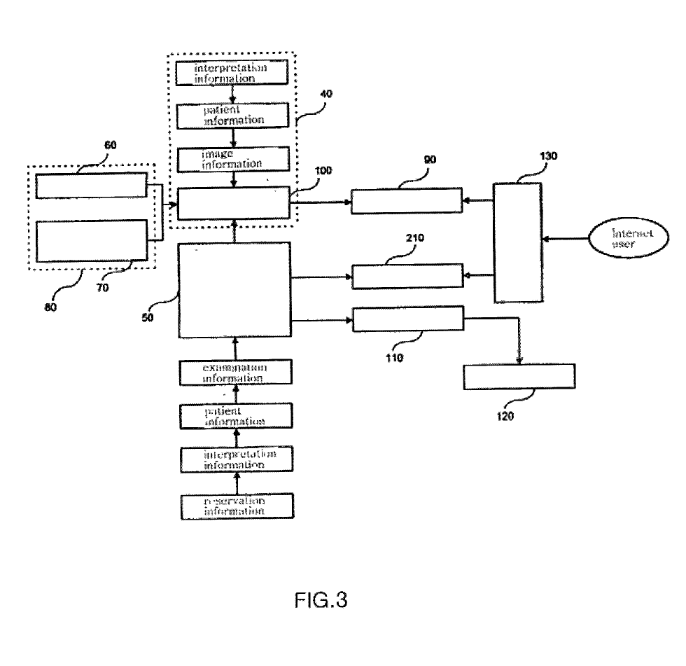

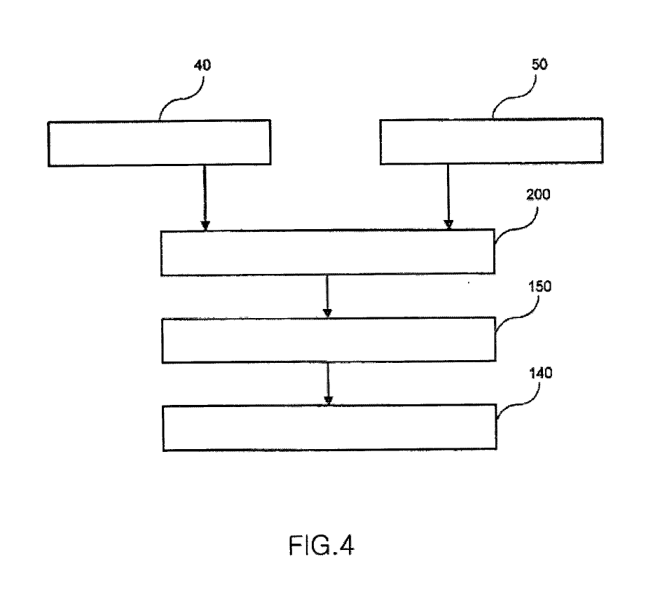

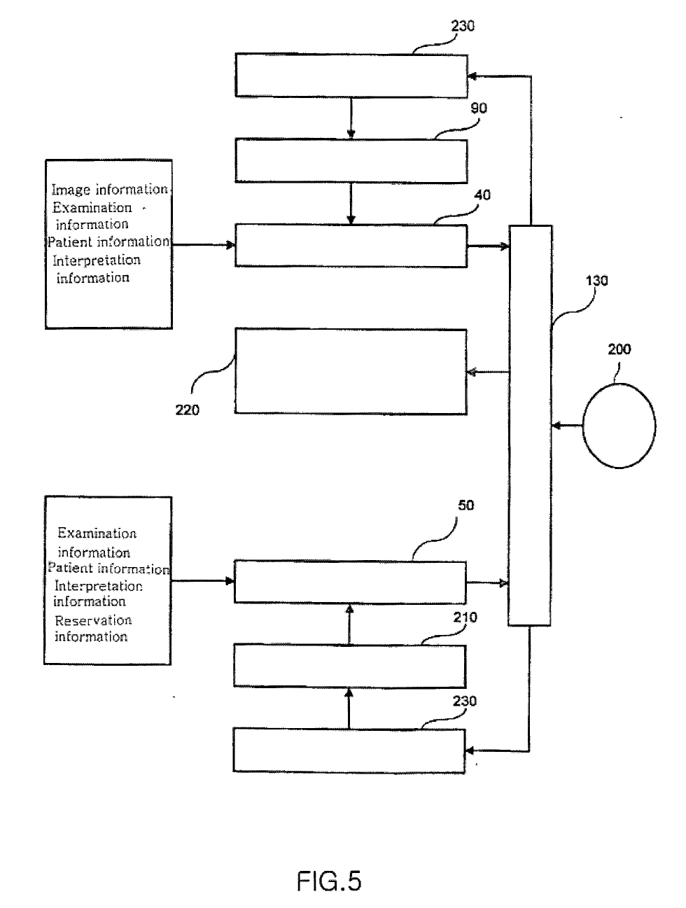

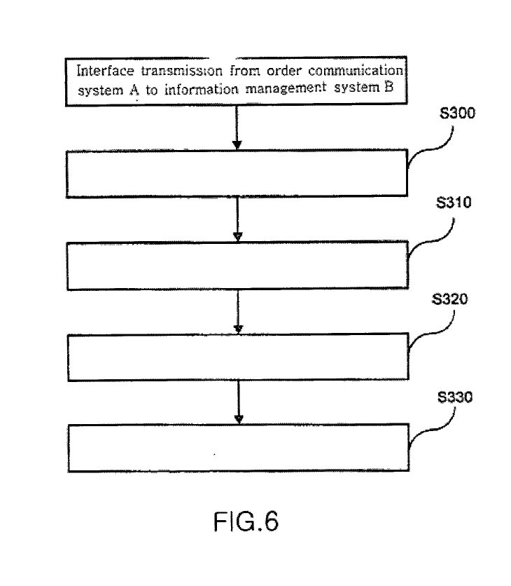

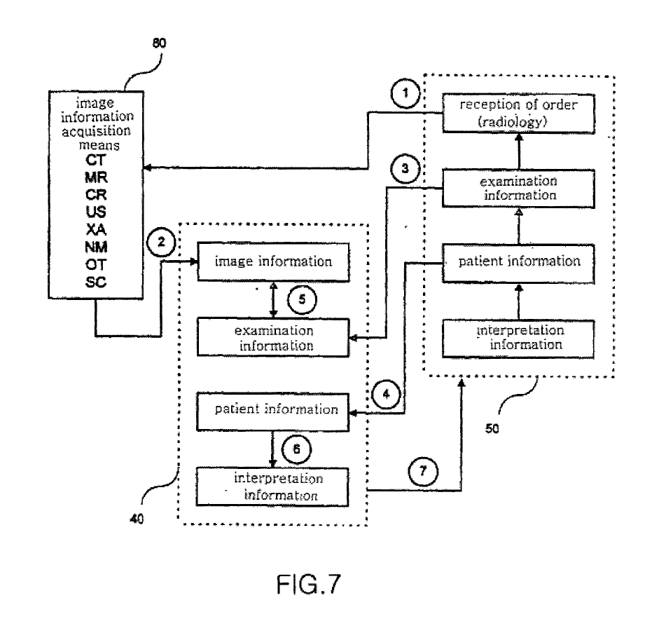

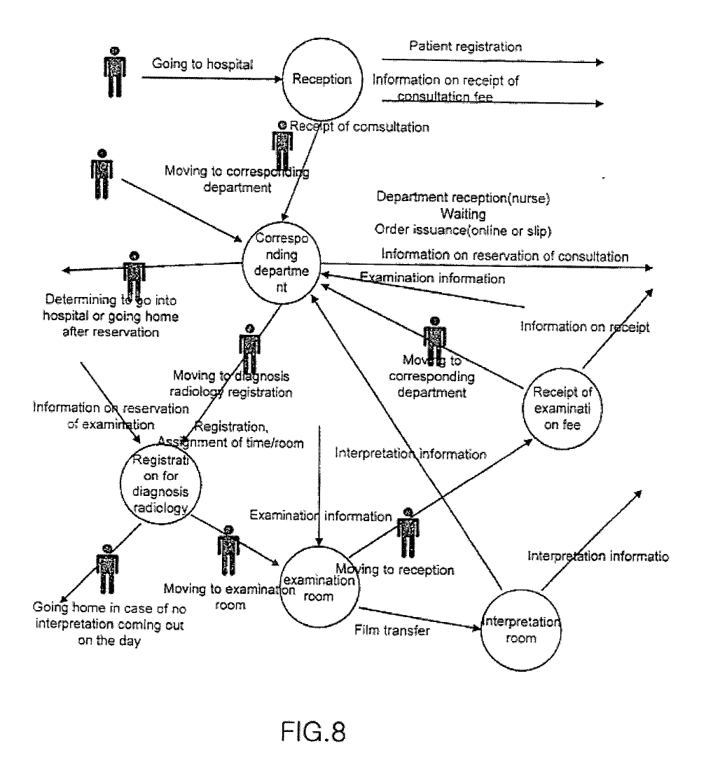

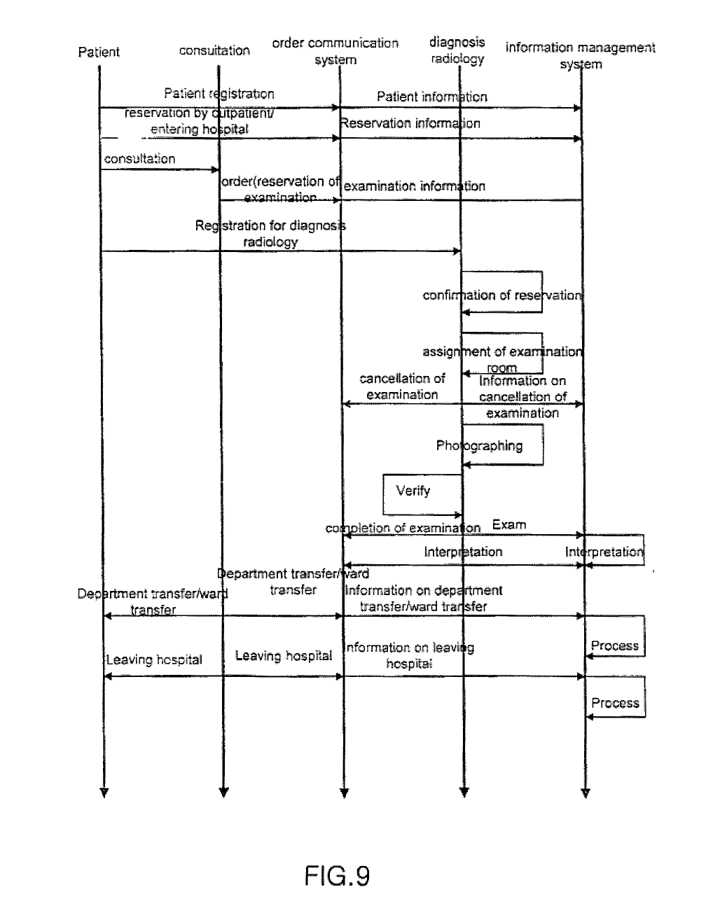

The present invention will now be described in detail in connection with preferred embodiments with reference to the accompanying drawings. FIG. 3 illustrates the configuration of a system according to the present invention, FIG. 4 illustrates an essential part of the system according to the present invention, FIG. 5 illustrates an embodiment of the system according to the present invention, FIG. 6 illustrates a message information processing flow according to the interface module of the present invention, FIG. 7 illustrates an information processing sequence according to the present invention, FIG. 8 illustrate a modeling of hospital works for analysis of the hospital works, and FIG. 9 is a sequence diagram showing order, admission and discharging and transfer processes with the lapse of time, processed by the present invention.

FIGS. 3 and 4 illustrate inter-operability between an information management system 40 and an order communication system 50 which provide integrated image and order information to a user terminal corresponding to an image observation part.

The present invention provides a hospital information management system constructed of the order communication system 50 and information management system 40, which are connected on a network 150. The information management system 40 includes: a means of acquiring image information 80 with an image photographing device 60 corresponding to an imaging system and an image control and compression device 70 for controlling the image photographing device 60, realizing data obtained from the image photographing device as images and compressing image data if required; and a host computer system 100 for inputting image information into a database 90 through the image information acquisition means 80. The order communication system 50 includes an image display 110 that requests image data from the host computer system 100 and combines or lays out images sent from the host computer system, a monitor 120 for monitoring the image made by the image display 110, and a web server 140 for transmitting data processed by the host computer system 100 to a user terminal 130 via the Internet.

Referring to FIGS. 3, 4 and 5, the aforementioned system includes an interface module 200 providing inter-operability with respect to information exchange between the information management system 40 and order communication system 50, a data interchanging unit 220 for inducing examination information and patient information, which are generated from examination images and stored in the database 90 of the information management system 40, interpretation information attained from the examination images, and patient information, examination information and reservation information to be reviewed through the respective systems via the interface module 200, and a result inputting unit 230 for storing the reviewed result processed by the data interchanging unit 220 in the databases 90 and 210 of the information management system 40 and order communication system 50.

When the user requests information through the user terminal 130 corresponding to the image observation part, a user application, via the network 150, he can access various data items (image information, examination information, patient information and interpretation information of the information management system 40) through the interface module 200 and, simultaneously review items (examination information, patient information, interpretation information and reservation information) of the order communication system 50.

Data processing in the interface module 200 is roughly divided into a step of transmitting information from the order communication system 50 to the information management system 40, and a step of integrating a process of transmitting information from the information management system 40 to the order communication system 50.

To illustrate, let the order communication system 50 be A and the information management system 40 be B. The process of transmitting information from A to B, as shown in FIG. 6, includes: a patient information message designing step S300 of changing the access path of patient images according to admission/discharging of patients and varying a patient searching path according to department transfer/ward transfer; an examination information message designing step S310 of, according to the path of information required for matching the photographed image of a patient and corresponding examination between A and B has an error or not, or the probability the information is defect-free, determining the matching; a medical treatment reservation message designing step S320 and an examination reservation information designing step S330 of, when a reservation of patient has been completed, setting it as a point of time at which the patient information message and examination information message are transmitted and, when the reservation is cancelled, delivering the result.

Information transfer from the information management system B to the order communication system A is carried out in such a manner so that when interpretation information is transmitted, reports on examinations and corresponding images match with the corresponding examinations and images, having creation, up-date and confirmation as points of time of information transmission.

Accordingly, the information management system 40 and order communication system 50 provide optimized review information requested from the interface module 200 to the user through their databases 90 and 210 while storing information in the databases so that they can exchange information and use shared databases 90 and 210.

The point of time of information transmission is determined according to a point of time of inputting reviewed information. For example, the point of time of transmitting information in the patient message-designing step corresponds to the time when a patient is newly registered, enters or leaves a hospital as well as when then are transferred to a different department or ward or come under the care of a different chief physician. The point of time of transmitting information in the examination information message-designing step corresponds to the time when a patient registers for the diagnosis radiology and when an examination in an examination room is cancelled.

The information transmission is performed, as shown in FIG. 7, through the first step of sending required information to the image information acquisition means 80 according to the photographing order of the order communication system 50, the second step of transmitting image information including order information received from the order communication system 50 to the information management system 40, the third step of transferring the order information of the order communication system 50 to the information management system 40, the fourth step of delivering patient information of the order communication system 50 to the information management system 40, the fifth step of updating the image information and examination information transferred from the image information acquisition means 80 and order communication system 50 to the information management system 40 through matching process, the sixth step of a doctor's using the image information to perform interpretation, examination information and patient information of the information management system 40, and the seventh step of transmitting interpretation information of the information management system 40 to the order communication system 50.

In cases where the point of time of transmitting information is determined according to the operation of each work, as described above, review information the user requires can be provided in an optimized state and, simultaneously, a large amount of the quantity of review information concentrated on the order communication system 50 is distributed to the information management system 40 to prevent network overload, improve stability of information and prepare for system down-time or transmission of error information.

Hospital information systems have a vast amount of contents and therefore, continuous upgrade of information occurs. The hospital system operates according to medical examination and treatment systems. To effectively support examination and treatment by the information management system constructed in the hospital according to the flow of the hospital operations, the interface between the order communication system and main computer system is necessary. It is vital to the operation of the hospital that events can be analyzed according to the generation and variation of data.

FIG. 8 illustrates a modeling of general hospital tasks ranging from registration of patient to reservation of the next consultation. The integrated hospital management system is capable of allowing inter-operability between the order communication system and information management system, from which and effective use of the two systems can be constructed. Furthermore, in order to construct an integrated system by which a single station can access both the order communication system 50 and information management system 40 to directly review images with respect to a treatment and interpretation result for that images through an order program of the order communication system 50, an interface with the order communication system 50 must be accurately designed. This needs not only the analysis of the order communication system 50 but also inter-operability of the order communication system with various information systems.

The present invention provides designing and realization of the integrated hospital management system. With the integrated hospital management system of the present invention, the flow of patient information, examination information, image data and interpretation information is analyzed by the interface between the information management system 40 and the order communication system 50 varying in real time. Simultaneously, new information is created through the interface module 200 and hospital operations are analyzed to maintain an organic relationship among departments.

Furthermore, the system of the present invention supports the construction of patient folders or interpretation-related folders, designing of hanging protocol and construction and review of image data suitable for routine round, confirmation and operation. Moreover, it is possible to support workflow and integrate hospital information systems. The user can grasp various information items according to his workflow and increase efficiency of medical examination and treatment. For instance, ordering, registration of examination, completion of examination, acquisition and review of images and checking the generation of interpretation result through the order communication system 50 can be processed according to flow of works and the user is informed of the processed result.

FIG. 9 is a sequence diagram of order/admission, discharging and transfer processes with the lapse of time. Table 1 represents construction of events depending on classification of information and inter-operability between the order communication 50 and information management system 40 according to information contents to be transmitted.

TABLE 1

| | | Contents of | Order | Information |
|---|---|---|---|---|
| Classification | | information to be | communication | management |
| of information | Events | transmitted | system | system |
| Patient | Registration | Patient's number, | Load information | Poll and process |
| information | | department, ward, | (record) into | message queue |
| | | examination code, | message queue | |
| | | (patient's) name, | | |
| | | date of (patient's) | | |
| | | birth, sex of patient | | |
| | Admission/ | Patient's number, | Load information | Poll and process |
| | Discharging | admission/discharging | (record) into | message queue |
| | | | message queue | |
| | Department | Patient's number, | Load information | Poll and process |
| | transfer/ward | department transfer/ | (record) into | message queue |
| | transfer | ward transfer | message queue | |
| Examination | Examination | Order number, patient | Load information | Poll and process |
| information | issuance/regi- | ID, registration date, | (record) into | message queue |
| | stration | registration time, | message queue | |
| | | department, ward, | | |
| | | examination code, | | |
| | | patient's name, | | |
| | | date of (patient's) | | |
| | | birth, sex (of patient), | | |
| | | examination item code, | | |
| | | examination item | | |
| Examination | Cancellation | Order number, | Load information | Poll and process |
| information | of | cancellation | (record) into | message queue |
| | examination | | message queue | |
| | Completion | Order number, | Load information | Poll and process |
| | of | examination | (record) into | message queue |
| | examination | execution time | message queue | |
| Interpretation | Write | Report number, patient's | Poll and process | Load |
| information | Transfer | number, registration date, | message queue | information(record) |
| | Read | interpretation input date, | | into message queue |
| | Confirm | interpretation input time, | | |
| | | interpretation doctor, | | |
| | | confirmation, classification | | |
| | | of interpretation, | | |
| | | examination item code, | | |
| | | examination item name | | |

In case the information classification is ‘patient information’, event is ‘registration’, and the contents of information to be transmitted include patient number, department, ward, code of examination and treatment, patient's name, date of birth, sex, as shown in table 1 when information is loaded into the message queue of the order communication system 50, the information management system 40 polls the message queue to process it. Similarly, the information management system polls the message queue to process it according to information classification and the contents of an event, or loads information into the message queue.

As described above, the present invention provides database interface between the information management system and the order communication system to allow information exchange between the two systems to be carried out at the precise point in time. This increases doctors' efficiency. In addition, the information management system or order communication system is separated from the other system in order to perform normal operation while the other system is erroneously operated, to prevent interruption of the system. Moreover, review information concentrated on one system is distributed to prevent system down-time due to network overload. Also, distributed databases can exchange information with each other in a timely manner to improve accuracy in the management of information in hospital environments, which require management of a large amount of information.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

What is claimed is:

1. An integrated hospital information management system having an order communication system and an information management system which are connected on a network, the information management system including: an image information acquisition means having an image photographing device corresponding to an imaging system and an image control and compression device for controlling the image photographing device, realizing data obtained from the image photographing device as images and compressing image data if required; and a host computer system for inputting image information into a database through the image information acquisition means, the order communication system including an image display that requests image data from the host computer system and combines or lays out images sent from the host computer system, a monitor for monitoring the image made by the image display, and a web server for transmitting data processed by the host computer system to a user terminal via the Internet,
the integrated hospital information management system comprised of:
an interface module providing inter-operability with respect to information exchange between the information management system and the order communication system;
a data interchanging unit for inducing examination information and patient information, which are generated from examination images and stored in the database of the information management system, interpretation information attained from the examination images, and patient information, examination information and reservation information to be reviewed through the respective systems via the interface module; and
a result-inputting unit for storing the reviewed result processed by the data interchanging unit in the databases of the information management system and the order communication system.

2. An integrated hospital information management method for maintaining compatibility and permitting the exchanging and sharing of various items of information including those pertaining to patients, images, reservations and examination, which exist on both an order communication and an information management system, the method including a step of transmitting information from the order communication system to the information management system and a step of integrating a process of transmitting information from the information management system to the order communication system,
the step of transmitting information from the order communication system to the information management system comprised of:
a patient information message designing step of changing an access path of patient images according to admission/discharging of patients and varying a patient searching path according to department transfer/ward transfer;
an examination information message designing step of, according to the path of information required for matching the patients' photographed image and corresponding examination between the order communication system and the information management system has an error or not or as the accuracy of information is guaranteed or not, determining the matching;
a medical treatment reservation message-designing step and an examination reservation information designing step which would, when a patient's reservation has been completed, set it as a point of time at which the patient information message and examination information message are transmitted and, when the reservation is cancelled, delivering the result, the process of transmitting information from the information management system to the order communication system being carried out in such a manner that interpretation information is transmitted, with reports on examinations and corresponding images matching with the corresponding examinations and images, having creation, up-date and confirmation as points of time of information transmission.

3. The method as claimed in claim 2, wherein the information transmission between the order communication system and the information management system is carried out through the first step of sending required information to the image information acquisition means according to photographing order of the order communication system,
the second step of transmitting image information including order information received from the order communication system to the information management system,
the third step of transferring the order information of the order communication system to the information management system,
the fourth step of delivering patient information of the order communication system to the information management system,
the fifth step of updating the image information and examination information transferred from the image information acquisition means and order communication system to the information management system through matching process,
and the sixth step of performing interpretation by a doctor taking charge of interpretation using the image information, examination information and patient information of the information management system, and the seventh step of transmitting interpretation information of the information management system to the order communication system.

4. The method as claimed in claim 2, wherein the point of time of transmitting information in the patient message-designing step corresponds to when a patient is newly registered, enters or leaves a hospital, or is transferred to a new department or ward or comes under the care of a different chief physician.

5. The method as claimed in claim 2, wherein the point of time of transmitting information in the examination information message-designing step corresponds to when a patient registers for the diagnosis radiology and when an examination in an examination room is cancelled.
